(19) United States
(12) Patent Application Publication
(10) Pub. No.: US 2014/0121618 A1
(43) Pub. Date: May 1, 2014

(54) Ergonomic System for Drainage and Disposal of Bodily Fluids

(71) Applicant: KIMBERLY-CLARK WORLDWIDE, INC., Neenah, WI (US)

(72) Inventors: Joseph A. Cesa, Cumming, GA (US); Adrienne A. Hershey, Cumming, GA (US); Stephen A. Baratian, Roswell, GA (US); Alison S. Bagwell, Alpharetta, GA (US); Lisa A. Linton, Alpharetta, GA (US); Carl Aronson, Wauconda, IL (US)

(73) Assignee: KIMBERLY-CLARK WORLDWIDE, INC., Neenah, WI (US)

(21) Appl. No.: 13/663,765

(22) Filed: Oct. 30, 2012

(51) Int. Cl. A61M 27/00; B31B 13/02

(52) U.S. Cl. 604/327; 493/121

(57) ABSTRACT

There is provided an ergonomic container for draining fluid from the pleural space or peritoneal cavity of a patient. The container is desirably easy to open, easy to pour, easy to hold, easy to control and keep from falling on the floor, and that has graphics that communicate the use and disposal of the container in a relatively simple and easy to understand manner.

This disclosure relates to apparatus or equipment for draining fluid from a body cavity and methods of using such apparatus or equipment to drain fluid from a body cavity. More specifically, the disclosure relates to equipment or apparatus used for peritoneal and pleural drainage and methods of using such apparatus or equipment to perform peritoneal and pleural drainage.

Drainage catheters are commonly placed utilizing a tunneling procedure. Using fluoroscopic guidance, a guide wire introducer with needle is inserted through the abdominal or chest wall at the desired insertion site. The needle is removed while the guide wire introducer is left in place. An initial incision is made through the guide wire insertion site. A second incision is made 5-8 cm from the initial incision. A tunneler/catheter assembly is passed subcutaneously from the second incision down to and out through the incision at the guide wire insertion site until the polyester cuff on the catheter lies about 1 cm inside the second incision. The insertion site is dilated; a peel away introducer sheath is threaded over the guide wire and advanced into the peritoneal space and pleural cavity. The guide wire and dilator are removed as a unit and the peel-away introducer sheath is left in place. The fenestrated end of the catheter is advanced into the sheath until all the fenestrations are within the peritoneal cavity and pleural cavity. This can be verified under fluoroscopy as fenestrations are located along the barium sulfate stripe. The peel-away sheath is removed and the incision is closed at the insertion site. The catheter is then typically sutured to the skin superior to the second incision. Variations to the tunneling procedure can be performed (retrograde, antegrade, over the wire). However; all procedures utilize two incisions and the tunneling technique.

Once a catheter has been placed and the surgery is complete, draining of the fluid may begin. The catheter is connected to a drainage container, which may be a vacuum bottle as described in US patent publication 2004/116902, for example. More commonly however, a relatively simple collapsible bag is used. Since patients having drainage catheters are generally ambulatory, many users/patients must be responsible for handling and disposing of the fluids that are drained. Current solutions involve users tearing and pouring fluids out of drainage bags themselves prior to disposing of the used bag. Existing drainage bags are difficult to open and hold during disposal, and as a result may expose the patient or a caregiver to these fluids as they dispose of them.

It would be desirable to have a collection container that is easy to use for patients, some of whom do not have the dexterity and coordination to use existing bags efficiently and comfortably.

### SUMMARY

The present disclosure addresses the problems described above by providing an ergonomic container for draining fluid from the pleural cavity or abdomen of a patient. The container is desirably easy to open, easy to pour, easy to hold, easy to control and keep from falling on the floor, and that has graphics that communicate the use and disposal of the container in a relatively simple and easy to understand manner. The ergonomic container is desirably used for pleural or peritoneal drainage procedures and is desirably a flexible bag.

The method may include the steps of: providing a drainage container assembly as generally described above; draining fluid utilizing the drainage tube and collecting the fluid in the collection container; disconnecting the container from the catheter, opening the container, pouring out the contents and disposing of the container properly.

Other objects, advantages and applications of the present disclosure will be made clear by the following detailed description.

### BRIEF DESCRIPTION OF THE DRAWINGS

These and other features of this disclosure will be more readily understood from the following detailed description of the various aspects of the disclosure taken in conjunction with the accompanying drawings in which:

FIG. 1 is an illustration of an exemplary improved drainage container showing a collapsible container having a fill opening for accommodating a drainage catheter, a pour spout with an easy-opening design, a handle, a hanging point and graphics that convey meaning to a user.

FIG. 2 is a graph of the amount of force needed to tear the material from which a bag is made. The scale on the left (Y-axis) represents the force needed to tear the material and the lower scale (X-axis) represents the strain in millimeters.

### DETAILED DESCRIPTION

Reference will now be made in detail to one or more embodiments, examples of which are illustrated in the drawings. It should be understood that features illustrated or described as part of one embodiment may be used with another embodiment to yield still a further embodiment. It is intended that the claims include these and other modifications and variations as coming within the scope and spirit of the disclosure.

The present disclosure addresses the problems described above by providing a collection container or bag for draining fluid from the pleural cavity or abdomen of a patient. The container is desirably easy to open, easy to pour, easy to hold, easy to control and keep from falling on the floor, and that has graphics (indicia) that communicate the use and disposal of the container in a relatively simple and easy to understand manner.

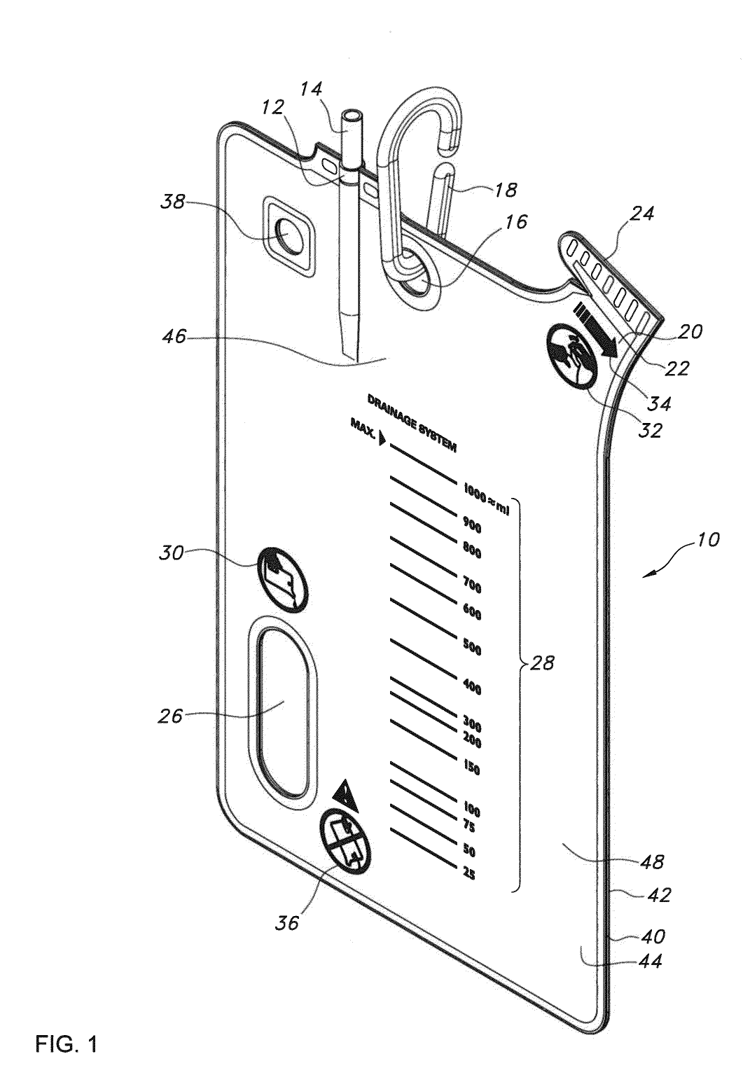

The collection container is desirably a collapsible bag with at least two opposing sides, as generally illustrated in FIG. 1. While the container is shown in FIG. 1 as generally rectangular, other shapes may be used and the drawing is not meant to limit the shape of the container in any way. In an aspect of the disclosure, a manual primer may be used to initiate flow into the drainage container. The primer could be (but is not limited to) a bulb or bellows configuration as conventionally known.

In one configuration the drainage container is a multi-layer bag with polyvinyl chloride as the main constituent. The sides 42, 44 are joined together, desirably continuously about the entire periphery 40, to form the container 10. This would help to ensure fluid containment in the event that the filled container 10 were dropped. Alternatively, the container may be made of a material, such as metallocene polyethylene (mPE) that would enable it to have superior tear resistance. A single layer mPE container offers advantages relative to polyvinyl chloride with respect to cut and tear resistance. In yet another configuration, a septum or membrane for a needle stick may be formed into the collapsible container. This septum should be self-sealing. For example, the septum may be made of a material such as halogenated butyl rubber (e.g., halo butyl rubber) and formulated or laminated so it is self-sealing.

The collection container 10 has an inlet 12 that is designed to accommodate a drainage catheter 14. The inlet 12 may be in the periphery 40 or may be in one of the sides 42, 44 of the collection container 10 but is in the upper part 46 of the container 10. In use, the catheter 14 is inserted into the container inlet 12 and flow may be begun using the optional primer (not shown) or by gravity.

The container 10 may be conveniently hung from, for example, the belt of the user, using any number of means for hanging. These means may include a piece (e.g. a hook) that is glued to the outer surface of one of the sides 42, 44 of the container, a clip having a hook with opposing members that close and tighten on either side of the container or a slit or hanging hole 16 in the upper part 46 of the container 10. As illustrated in a non-limiting example, a carabineer 18 may be inserted into the hanging hole 16 for use in suspending the container 10 above the ground. Any other suitable hanger may be used in place of the carabineer 18 in FIG. 1, as this is shown only for illustration. The hanging hole 16 is an opening through the container 10 inside the periphery 40 that has edges sealed in the same manner as the periphery 40 of the container 10. Hanging the container on the user's clothing or in another appropriate location is a surprisingly helpful aspect of this disclosure as it allows the user to be mobile while drainage occurs. Hanging the container also keeps the container away from unsanitary surfaces, such as it would be exposed to lying on the floor.

Once the container 10 is full or drainage through the catheter 14 is stopped, the catheter 14 may be removed from the inlet 12. The container 10 can be disconnected from its hanger (if a hanger was used) and drained. In order to drain the container 10, either the inlet 12 or the spout 20 may be used. If it is desired to use the inlet 12 to drain the container 10, the container 10 must be inverted over an appropriate disposal site and the fluid in the container 10 allowed to flow from the inlet 12 into the site. It is believed that drainage through the inlet 12 will be slower than using the spout 20.

The spout 20 is desirably located in the upper part 46 of the container 10 in a corner of the container 10 and is normally sealed during drainage. The spout 20 is formed when the tear tab 24 is removed from the container 10 by a user. Once the user removes the tear tab 24 from the container 10, the spout 20 will be formed and the container 10 will be open. The tear tab 24 will be discussed in greater detail below.

The container also desirably has a means for holding the container. This means for holding may be a roughened area of the outer surface of one or both sides of the container; the roughening providing an improved grip for a user's fingers. The holding means may be a handle 26 on a lower part 48 of the container 10 generally opposite the spout 20. The handle 26 is desirably an opening through the container 10 that has edges sealed in the same manner as the periphery 40 of the container 10. The user will desirably grasp the container 10 by its holding means and then pour the contents out of the spout 20 of the container 10 into an appropriate disposal site. If the holding means is a handle 26, this is done by the user inserting his fingers through the handle 26 and tipping the container 10 so that the fluid will pour out of the spout 20.

The container 10 is not intended for re-use and should be disposed of or recycled properly. While the container is flexible and will expand and collapse as it is being filled and drained, a vent 38 is provided in the upper part 46 of the container 10 for air to move out of the container as it is being filled and into the container as it is being drained. This vent 38 does not allow liquid to pass and is of conventional construction as known in the art.

As noted above, the container 10, hanging hole 16, vent 28, and handle 26 are sealed about their respective peripheries. This may be done through known means of sealing polymers like radio frequency (RF) welding, ultrasonic welding, thermal welding and laser welding. Any method may be used provided the weld is sufficiently strong and does not allow fluid to leak.

As may be seen in FIG. 1, the tear tab 24 is formed by the two (un-joined) sides 42, 44 or layers of the container 10. The tear tab 24 forms a line of weakness that may be torn by the application of a relatively low amount of force, since the users of this container 10 may not have normal strength. It should be noted that the opposing sides 42, 44 of the container 10 are not bonded together at the tear tab 24. It has been found that the amount of force needed to tear the tab can be reduced and the propagation of the tear better controlled by merely treating each opposing side 42, 44 of the container 10 at the location of the tear tab 24 ultrasonically, thermally, with a laser or with radio frequency without actually welding the two sides 42, 44 together. I.e., the tear tab 24 is not formed by welding the two sides of the bag together but rather by the formation of a line of weakness on each side adjacent each other. This means that if the container 10 contains liquid and is tipped to move liquid toward the spout 20 before the tear tab 24 is removed, the liquid will pass beyond the tear tab 24, but the liquid will not escape from the container 10 since the periphery 40 of the container 10 is sealed.

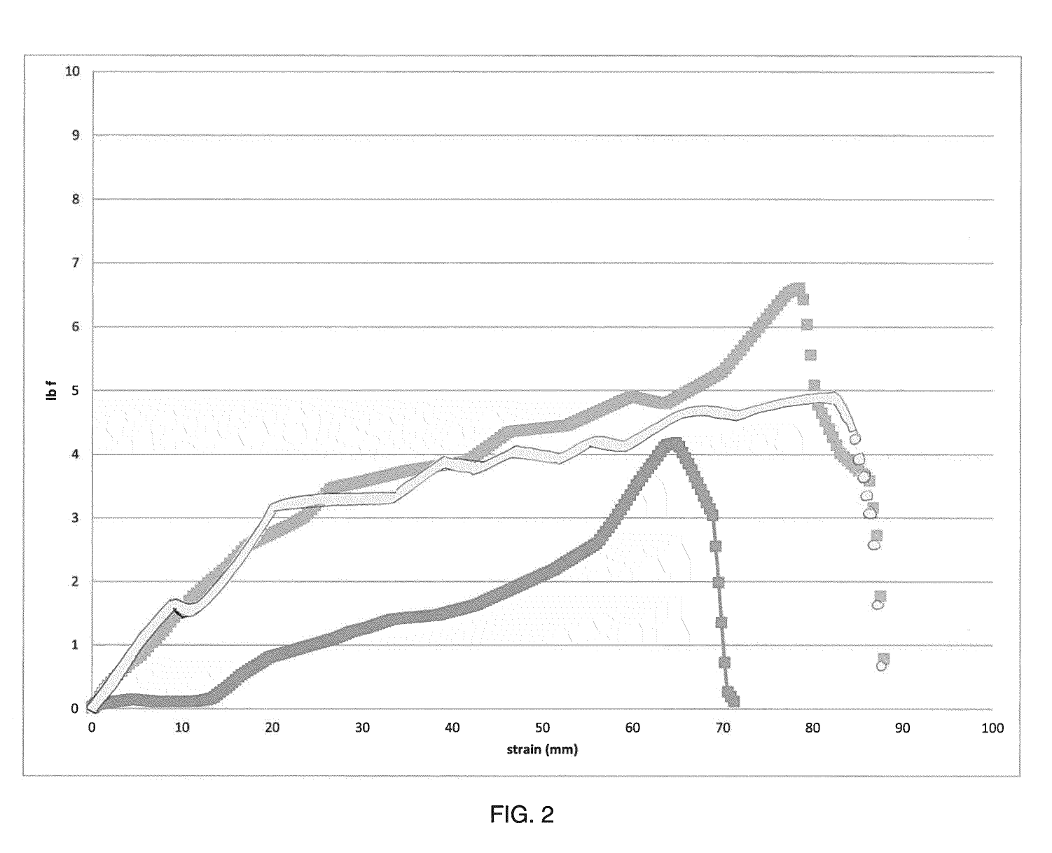

FIG. 2 is a graph of the amount of force needed to tear the material from which a bag is made. The scale on the left (Y-axis) represents the force needed to tear the material and the lower scale (X-axis) represents the strain in millimeters. The upper-most line represents the force needed to tear open a bag marketed by C.R. Bard, Inc. as a container for the Aspira® peritoneal drainage kit. The middle line represents the force needed to tear an exemplary bag, prior to any treatment to weaken the material. In this case the exemplary bag is made from two layers of polyvinyl chloride (PVC), one layer having a thickness of 12 mils (0.305 mm) and the other having a thickness of 10 mils (0.254 mm). The lowest line represents the amount of force needed to tear the exemplary bag after treatment by RF welding. The graph clearly shows that the treatment of the material results in a lower amount of force being necessary to tear the material than that needed to tear the C.H. Bard bag or the untreated bag used herein. The peak force needed to tear the treated, exemplary material is desirably at least 10 percent less than that of the Bard bag, desirably at least 25 percent less and more desirably at least a third less. The average pull force to tear the material was just over 3 lbf for Bard and the untreated material and the average pull force for the treated material was just under 1.5 lbf, or about half of the others. The disclosed method of producing the tear tab 24 (above) results in the amount of force required to remove the tear tab 24 being relatively low; desirably between about 2 and 5 lbf (8.9 and 22 Newtons), more desirably between 3 and 5 lbf (13.3 and 22.2 N) and still more desirably between 3.5 and 4.5 lbf (15.6 and 20 N).

Various indicia may be placed on at least one side 42, 44 of the container 10 to aid a user in the use of the container 10. For example and as illustrated in FIG. 1, the container 10 may be calibrated to show the volume of liquid contained. These calibrations 28 may aid in determining the state of the patient's condition and may aid in determining when it is necessary to use an additional container 10. A pouring illustration 30 may be included to show the user how to hold the container 10 by the handle 26 in order to pour the fluid from the spout 20. The area of the spout 20 may have a tearing illustration 32 and/or an illustration of an arrow 34 to show the user how to remove the tear tab 24 from the container 10 and to show the direction the fluid should be poured out of the container 10.

Other optional indicia may be used to warn the user against hanging the bag from the handle, which would turn the container 10 upside down, since this may cause leakage or prevent the bag from draining effectively. This leakage/drainage warning illustration 36 is shown in FIG. 1.

The dimensions of the container 10 may vary and still be within the bounds of the disclosure. The container 10, not including the spout 20, may have a length of from 8 to 18 inches (20 to 46 cm) and a width of from 4 to 10 inches (10 to 25 cm), though there are no reasons beyond design preference and volume requirements that the container 10 could not be larger or smaller. More particularly, the container 10 may have a length of from 10 to 13 inches (25 to 33 cm) and a width of from 5 to 9 inches (13 to 23 cm) and still more particularly the container 10 may have a length of about 11 inches (28 cm) and a width of about 7 inches (17.8 cm). The handle 26 is desirably large enough to fit a hand; about 2.5 inches (6.35 cm) in length and about 0.9 inches (2.3 cm) in width. The spout 20 desirably is about an inch (2.5 cm) wide when opened and the container 10 desirably has a capacity of from 750 to 1250 ml, more particularly about 1000 ml. The vent 38 may have a diameter of about a half inch (1.25 cm) and the hanging hole 16 a diameter of about 0.6 inches (1.6 cm).

The present disclosure encompasses an improved drainage method (e.g., an improved pleural or peritoneal drainage procedure). The method includes the steps of: providing a drainage assembly as generally described above; draining fluid utilizing the drainage tube and collecting the fluid in the collection container; and disposing of the fluid.

While the present disclosure has been described in connection with certain preferred embodiments it is to be understood that the subject matter encompassed by way of the present disclosure is not to be limited to those specific embodiments. On the contrary, it is intended for the subject matter of the disclosure to include all alternatives, modifications and equivalents as can be included within the spirit and scope of the claims.

We claim:

1. A container for accepting fluid from the pleural space or peritoneal cavity of a patient, the container comprising:
two opposing sides forming said container, connected together about a periphery of said container;
an inlet in an upper part of the container that is designed to accommodate a drainage catheter;
a spout located in an upper corner of the container;
wherein said spout is formed when a tear tab is removed from the container along a line of weakness.

2. The container of claim 1 wherein said tear tab is formed by the two opposing sides of the container without welding the two sides together at the location of the tear tab.

3. The container of claim 2 wherein said line of weakness is produced by a method selected from the group consisting of radio frequency welding, ultrasonic welding, thermal welding and laser welding.

4. The container of claim 2 wherein said tear tab is removed by an amount of force between about 2 and 6 lbf (8.9 and 26.7 N).

5. The container of claim 2 wherein said tear tab is removed by an amount of force between 3 and 5 lbf (13.3 and 22.2 N).

6. The container of claim 2 wherein said tear tab is removed by an amount of force between 3.5 and 4.5 lbf (15.6 and 20 N).

7. The container of claim 1 wherein the assembly is configured for peritoneal and pleural drainage procedures.

8. The container of claim 1, wherein the container has a hanging hole in an upper part that may be used to hang the container during drainage.

9. The container of claim 1, wherein the container has a handle in a lower part, generally opposite said spout.

10. The container of claim 1, further comprising indicia to aid a user in use of the container.

11. The container of claim 1 wherein said container has a liquid capacity of from about 750 to 1250 ml.

12. A container for accepting fluid from the pleural space or peritoneal cavity of a patient, the container comprising:
two sides joined about a periphery to form said container;
indicia on at least one side of said container to aid a user in use of the container;
an inlet in an upper part of the container that is designed to accommodate a drainage catheter;
a hanging hole in an upper part that may be used to hang the container during drainage;
a spout located in an upper corner of the container and a handle in a lower part of said container, generally opposite said spout; and,
wherein said spout is formed when a tear tab is removed from the container along a line of weakness.

13. The container of claim 12 wherein said line of weakness is produced by a method selected from the group consisting of radio frequency welding, ultrasonic welding, thermal welding and laser welding.

14. A method of draining fluid from the pleural space or peritoneal cavity of a patient and disposing of said fluid, including the steps of:
providing a drainage container having an inlet in an upper part of the container that is designed to accommodate a drainage catheter, said container having a spout located in an upper corner of the container; said spout formed when a tear tab is removed from the container by a user;
draining fluid into said container; and,
disposing of the fluid by opening the spout by removing said tear tab at a line of weakness, holding said container by a handle located generally opposite said spout and pouring said fluid out of said container into an appropriate site.
